(19) United States
(12) Patent Application Publication
(10) Pub. No.: US 2023/0181233 A1
(43) Pub. Date: Jun. 15, 2023

(54) TREATMENT TOOL

(71) Applicant: Olympus Corporation, Tokyo (JP)

(72) Inventors: Akira MIYAZAKI, Yokohama-shi (JP); Kazuhiro Tanaka, Hachioji-shi (JP); Ojiro Kitamura, Hachioji-shi (JP)

(21) Appl. No.: 18/167,558

(22) Filed: Feb. 10, 2023

Related U.S. Application Data
(63) Continuation of application No. PCT/JP20/31907, filed on Aug. 24, 2020.

(51) Int. Cl. A61B 18/08

(52) U.S. Cl. CPC A61B 18/085; A61B 2018/00601

(57) ABSTRACT

A treatment tool includes: a tubular insertion tube; an end effector that is provided at a distal end of the insertion tube and is bendable with respect to the insertion tube, the end effector being configured to apply treatment energy to a living tissue according to supplied power to treat the living tissue; a shaft body that is provided in the end effector and has an outer peripheral surface located on a circumference of a specific circle centered on a rotation axis when viewed from a direction along the rotation axis, the rotation axis allowing the end effector to be bent with respect to the insertion tube; and a wiring that is inserted through the insertion tube and serves as a supply path of the power, the shaft body including a passage through which the wiring is inserted.

### CROSS-REFERENCE TO RELATED APPLICATION

This application is a continuation of International Application No. PCT/JP2020/031907, filed on Aug. 24, 2020, the entire contents of which are incorporated herein by reference.

### BACKGROUND

### 1. Technical Field

The present disclosure relates to a treatment tool.

### 2. Related Ar1

In the related art, there has been known a treatment tool that can bend (swing) an end effector that treats a living tissue by applying treatment energy to the living tissue (see, for example, WO 2017/043128 A).

### SUMMARY

In some embodiments, a treatment tool includes: a tubular insertion tube at least a part of which is configured to be inserted into a body; an end effector that is provided at a distal end of the insertion tube and is bendable with respect to the insertion tube, the end effector being configured to apply treatment energy to a living tissue according to supplied power to treat the living tissue; a shaft body that is provided in the end effector and has an outer peripheral surface located on a circumference of a specific circle centered on a rotation axis when viewed from a direction along the rotation axis, the rotation axis allowing the end effector to be bent with respect to the insertion tube; and a wiring that is inserted through the insertion tube and serves as a supply path of the power, the shaft body including a passage through which the wiring is inserted.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of a treatment tool according to a first embodiment;

FIG. 2 is a diagram illustrating the configuration of the treatment tool according to the first embodiment;

FIG. 3 is a diagram illustrating the configuration of the treatment tool according to the first embodiment;

FIG. 4 is a diagram illustrating the configuration of the treatment tool according to the first embodiment;

FIG. 5 is a diagram illustrating the configuration of the treatment tool according to the first embodiment;

FIG. 6 is a diagram illustrating the configuration of the treatment tool according to the first embodiment;

FIG. 7 is a diagram illustrating a connection structure between an end effector, a sheath, a bending mechanism, and an opening/closing mechanism;

FIG. 8 is a diagram illustrating the connection structure between the end effector, the sheath, the bending mechanism, and the opening/closing mechanism;

FIG. 9 is a diagram illustrating the connection structure between the end effector, the sheath, the bending mechanism, and the opening/closing mechanism;

FIG. 10 is a diagram illustrating the connection structure between the end effector, the sheath, the bending mechanism, and the opening/closing mechanism;

FIG. 11 is a diagram illustrating the connection structure between the end effector, the sheath, the bending mechanism, and the opening/closing mechanism;

FIG. 12 is a diagram illustrating a joint structure between first and second wires and first and second bending rods;

FIG. 13 is a diagram illustrating a modification of the joint structure between the first and second wires and the first and second bending rods;

FIG. 14 is a diagram illustrating a modification of the joint structure between the first and second wires and the first and second bending rods;

FIG. 15 is a diagram for describing a change in driving force in an opening/closing rod and a link mechanism;

FIG. 16 is a diagram for describing the change in driving force in the opening/closing rod and the link mechanism;

FIG. 17 is a diagram for describing the change in driving force in the opening/closing rod and the link mechanism;

FIG. 18 is a diagram for describing the change in driving force in the opening/closing rod and the link mechanism;

FIG. 19 is a diagram for describing the change in driving force in the opening/closing rod and the link mechanism;

FIG. 20 is a diagram illustrating a distal end portion of a treatment tool according to a second embodiment;

FIG. 21 is a diagram illustrating the distal end portion of the treatment tool according to the second embodiment;

FIG. 22 is a diagram illustrating the distal end portion of the treatment tool according to the second embodiment;

FIG. 23 is a diagram illustrating a shaft body according to a third embodiment;

FIG. 24 is a diagram illustrating a shaft body according to a fourth embodiment; and

FIG. 25 is a diagram illustrating a configuration of a medical device according to a fifth embodiment.

### DETAILED DESCRIPTION

Hereinafter, modes for carrying out the disclosure (hereinafter, referred to as embodiments) will be described with reference to the drawings. The disclosure is not limited by the embodiments described below. In the description of the drawings, the same portions are denoted by the same reference numerals.

### First Embodiment

Schematic Configuration of Treatment Tool

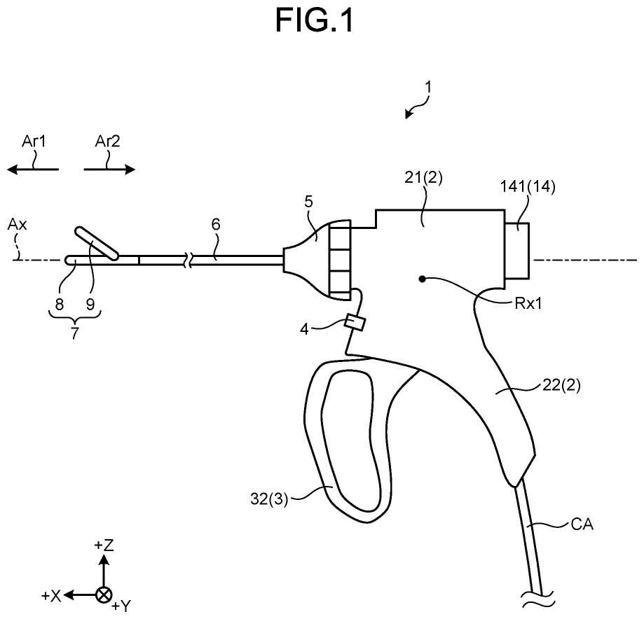

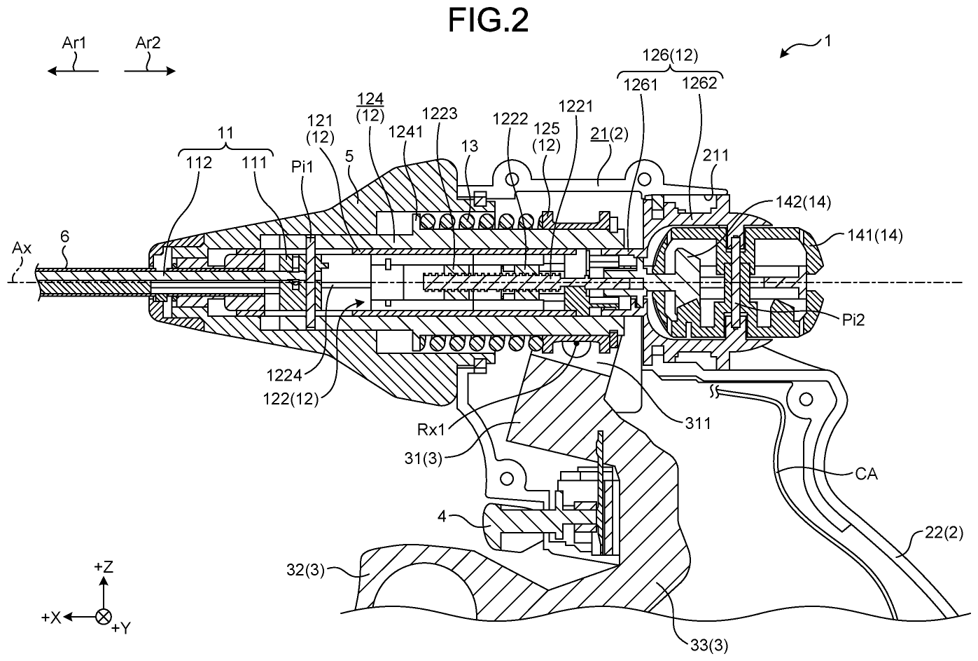

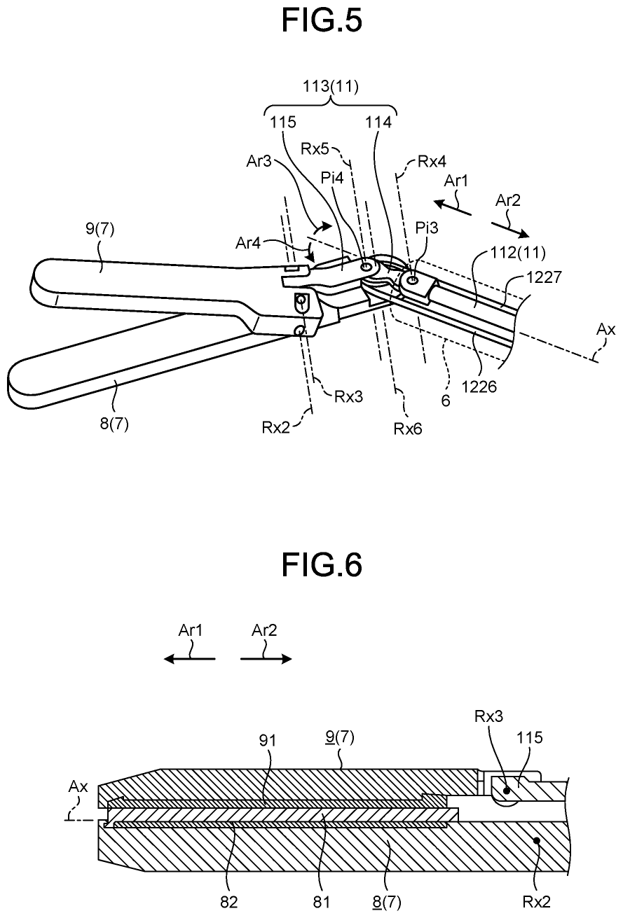

FIGS. 1 to 6 are diagrams illustrating a configuration of a treatment tool 1 according to the first embodiment. Specifically, FIG. 1 is a diagram illustrating an overall configuration of the treatment tool 1. FIGS. 2 to 4 are diagrams illustrating a configuration in a housing 2. FIG. 5 is a diagram illustrating a distal end portion of the treatment tool 1. FIG. 6 is a cross-sectional view of the distal end portion of the treatment tool 1 taken along a plane including a central axis Ax (FIG. 1) of a sheath 6.

In FIGS. 1 and 2, XYZ coordinate axes of an X axis, a Y axis, and a Z axis orthogonal to each other are used. The X axis is an axis parallel to the central axis Ax. The Y axis is an axis orthogonal to the paper surface of FIGS. 1 and 2. The Z axis is an axis along a vertical direction in FIGS. 1 and 2. Hereinafter, one side (+X-axis side) along the central axis Ax is referred to as a distal end side Ar1, and the other side (−X-axis side) is referred to as a proximal end side Ar2.

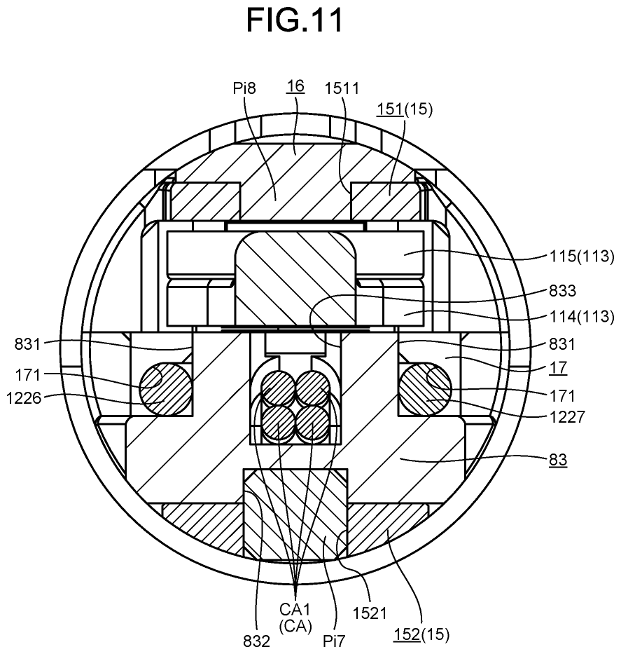

The treatment tool 1 treats a treatment target site (hereinafter, referred to as a target site) by applying treatment energy to the target site in a living tissue. As the treatment, coagulation and incision of the target site can be exemplified. As illustrated in FIGS. 1 to 6, the treatment tool 1 includes a housing 2 (FIGS. 1 and 2), a movable handle 3 (FIGS. 1 and 2), a switch 4 (FIGS. 1 and 2), a rotary knob 5 (FIGS. 1 to 4), a sheath 6 (FIGS. 1 to 3 and 5), an end effector 7 (FIGS. 1, 5, and 6), an electric cable CA (four electrically conductive wires CA1 (see FIG. 11), a rotating member 12 (FIGS. 2 to 4), a coil spring 13 (FIGS. 2 to 4), and a bending operating unit 14 (FIGS. 1 to 4).

The housing 2 supports the entire treatment tool 1. As illustrated in FIG. 1 or 2, the housing 2 includes a substantially cylindrical housing body 21 coaxial with the central axis Ax, and a fixed handle 22 extending from the housing body 21 toward a −Z-axis side (lower side in FIGS. 1 and 2) and gripped by an operator.

As illustrated in FIG. 1 or 2, the movable handle 3 includes a handle base 31 (FIG. 2), a handle body 32, and a handle connection portion 33 (FIG. 2).

As illustrated in FIG. 2, the handle base 31 is located in the housing 2. A portion of the handle base 31 on a +Z axis side (upper side in FIGS. 1 and 2) is pivotally supported to the housing 2 so as to be rotatable about a first rotation axis Rx1 (FIGS. 1 and 2). A pair of engaging portions 311 (FIG. 2), which protrude toward the +Z axis side in a bifurcated state and face each other along a Y axis direction in a state of sandwiching a slider 125 (FIG. 2) constituting the rotating member 12, is provided at an end of the handle base 31 on the +Z axis side. The pair of engaging portions 311 engage with the slider 125. In FIG. 2, only the engaging portion 311 in the +Y axis direction (depth direction of the paper surface of FIG. 2) is illustrated in the pair of engaging portions 311.

The handle body 32 receives a closing operation and an opening operation by the operator, and is located outside the housing 2 as illustrated in FIG. 1 or 2.

As illustrated in FIG. 2, the handle connection portion 33 is disposed across the inside and the outside of the housing 2 and connects the handle base 31 and the handle body 32.

As illustrated in FIG. 1 or 2, the switch 4 is provided in a state of being exposed to the outside from a side surface of the fixed handle 22 on the distal end side Ar1, and receives an output start operation by the operator. The output start operation is an operation of pressing the switch 4, and is an operation of starting application of the treatment energy to the target site. Then, the switch 4 outputs an operation signal corresponding to the output start operation to an external control device (not illustrated) via the electric cable CA (FIGS. 1 and 2).

The rotary knob 5 has a substantially cylindrical shape extending along the central axis Ax, and is supported by the housing body 21 so as to be rotatable about the central axis Ax in a posture coaxial with the central axis Ax. The rotary knob 5 receives a rotation operation by the operator. By the rotation operation, the rotary knob 5 rotates about the central axis Ax with respect to the housing body 21.

The sheath 6 corresponds to an insertion tube. An end of the sheath 6 on the proximal end side Ar2 is inserted into the rotary knob 5, and is fixed to an inner surface of the rotary knob 5 by welding or the like. That is, the sheath 6 rotates about the central axis Ax together with the rotary knob 5 in response to the rotation operation of the rotary knob 5 by the operator.

As illustrated in FIG. 1, 5, or 6, the end effector 7 includes first and second graspers 8 and 9.

The first grasper 8 has an elongated shape extending along the central axis Ax.

As illustrated in FIG. 6, the first grasper 8 includes a first electrode 81 and a heater 82 at an end on the distal end side Ar1 facing the second grasper 9.

The first electrode 81 is formed of an electrically conductive material having high thermal conductivity such as copper, and has an elongated shape extending along the central axis Ax. The first electrode 81 is provided in the first grasper 8 in a state of being exposed to the outside.

The heater 82 is provided in the first grasper 8 in a state of being hidden inside by the first electrode 81. When electric power is supplied to the heater 82, the heater 82 generates heat and heats the first electrode 81. Examples of the heater 82 include a sheet heater in which an electrically conductive pattern is patterned on a substrate formed of polyimide or the like, a ceramic heater in which an electrically conductive pattern is patterned on a ceramic substrate such as aluminum nitride, and other printing heaters.

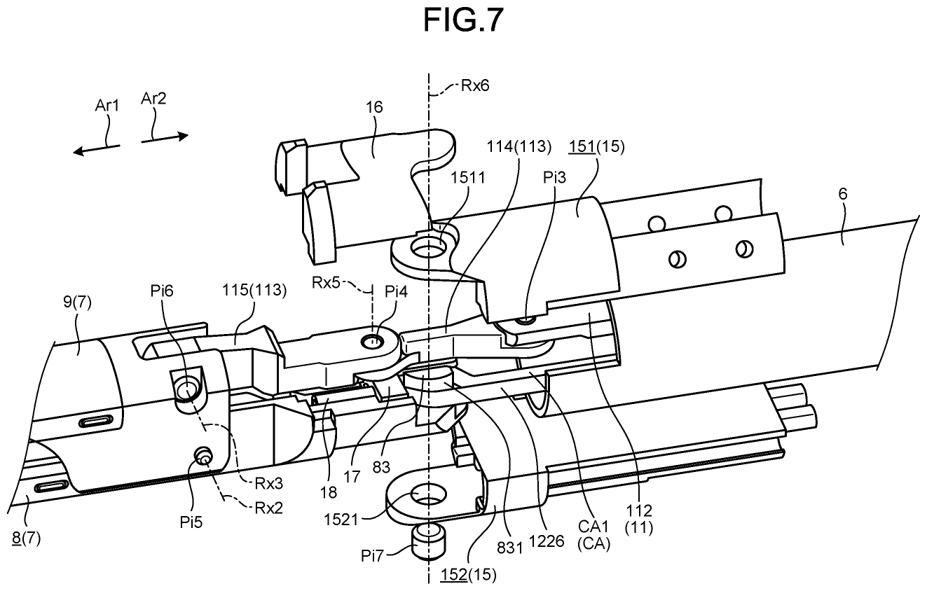

In the first grasper 8, a shaft body 83 (see FIG. 7) for bending (rotating) the end effector 7 with respect to the sheath 6 about a sixth rotation axis Rx6 (FIG. 5) is provided at the end on the proximal end side Ar2. The sixth rotation axis Rx6 corresponds to a rotation axis, and is an axis orthogonal to the central axis Ax. Furthermore, the sixth rotation axis Rx6 is an axis along an opening/closing direction (the vertical direction in FIG. 6) of the second grasper 9 with respect to the first grasper 8.

A detailed configuration of the shaft body 83 will be described in “Connection structure between end effector, sheath, bending mechanism, and opening/closing mechanism” described later.

The second grasper 9 has an elongated shape extending along the central axis Ax. A longitudinal length of the second grasper 9 is shorter than a longitudinal length of the first grasper 8. The second grasper 9 is pivotally supported with respect to the first grasper 8 so as to be rotatable about the second rotation axis Rx2 (FIGS. 5 and 6) by a columnar fifth pin Pi5 (see FIG. 7) having the end on the proximal end side Ar2 bridged between the first grasper 8 and the second grasper 9. When the second grasper 9 rotates about the second rotation axis Rx2, the second grasper 9 is opened and closed with respect to the first grasper 8, and the target site can be grasped between the first and second graspers 8 and 9.

In the second grasper 9, a portion facing the first electrode 81 is provided while a second electrode 91 (FIG. 6) formed of an electrically conductive material is exposed to the outside. The first and second electrodes 81 and 91 correspond to electrodes.

In the second grasper 9, a columnar sixth pin Pi6 (see FIG. 7) to which an opening/closing mechanism 11 constituting the rotating member 12 is connected is provided at the end on the proximal end side Ar2. The sixth pin Pi6 extends along a third rotation axis Rx3 parallel to the second rotation axis Rx2.

The four electrically conductive wires CA1 correspond to wirings. These four electrically conductive wires CA1 are electrically conductive wires constituting a part of the electric cable CA drawn out from an end of the fixed handle 22 on the −Z-axis side into the housing 2. The four electrically conductive wires CA1 are drawn out into the sheath 6 from the end of the fixed handle 22 on the −Z-axis side after following the inside of the housing 2 and the inside of the rotary knob 5. Among the four electrically conductive wires CA1, the two electrically conductive wires CA1 are electrically connected to the first and second electrodes 81 and 91. The remaining two electrically conductive wires CA1 are electrically connected to the heater 82.

An external control device (not illustrated) operates as described below in response to the output start operation to the switch 4 by the operator.

The control device supplies high-frequency power between the first and second electrodes 81 and 91 via the two electrically conductive wires CA1. As a result, a high-frequency current flows through the target site grasped between the first and second electrodes 81 and 91. In other words, high frequency energy is applied as treatment energy to the target site. Then, the target site is treated.

The control device supplies electric power to the heater 82 via the two electrically conductive wires CA1. As a result, the heat from the heater 82 is transmitted to the target site grasped between the first and second electrodes 81 and 91 through the first electrode 81. In other words, thermal energy is applied as the treatment energy to the target site. Then, the target site is treated.

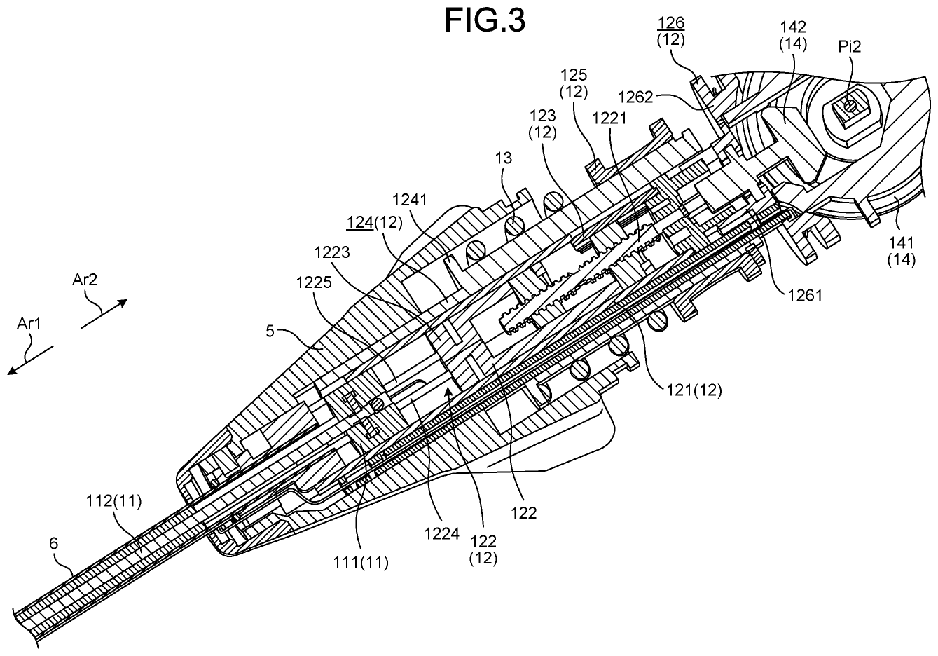

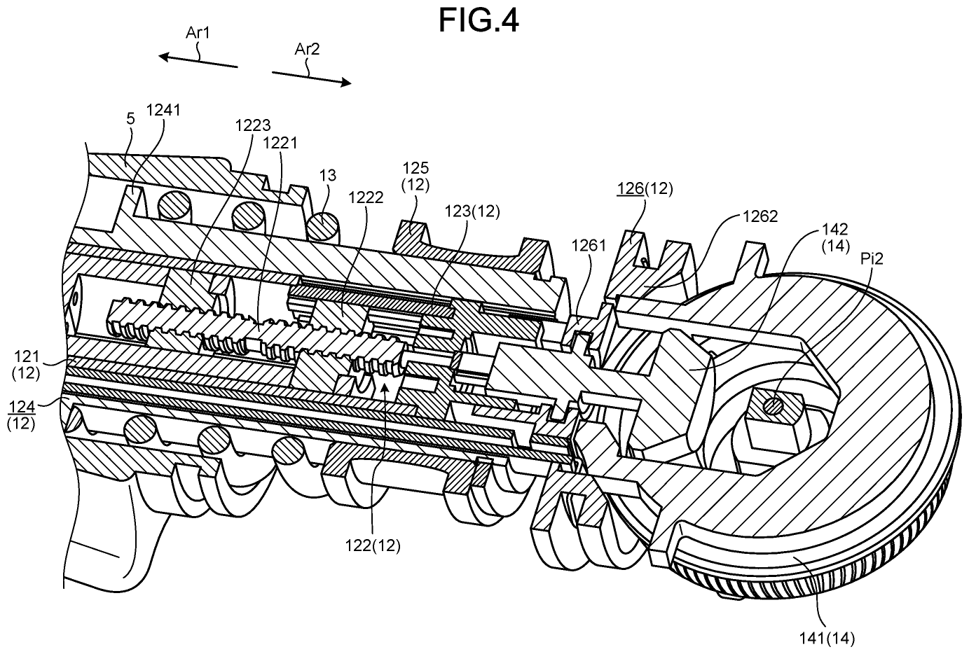

The rotating member 12 rotates about the central axis Ax together with the rotary knob 5 in response to the rotation operation of the rotary knob 5 by the operator. As illustrated in FIGS. 2 to 4, the rotating member 12 includes a first support member 121, a bending mechanism 122, a rotation restricting member 123 (FIGS. 3 and 4), a slider receiver 124, a slider 125, the opening/closing mechanism 11 (FIGS. 2 and 3), and a second support member 126.

As illustrated in FIGS. 2 to 4, the first support member 121 has a cylindrical shape extending along the central axis Ax and is disposed in a posture coaxial with the central axis Ax. More specifically, the first support member 121 is inserted through the rotary knob 5 and the housing body 21 in a state of straddling the rotary knob 5 and the housing body 21. An end of the first support member 121 on the distal end side Ar1 is fixed to the inner surface of the rotary knob 5 by welding or the like.

The first support member 121 described above supports a part of the bending mechanism 122 and a part of the opening/closing mechanism 11 inside thereof.

The configurations of the bending mechanism 122 and the rotation restricting member 123 will be described together with the configuration of the bending operating unit 14.

As illustrated in FIGS. 2 to 4, the slider receiver 124 has a cylindrical shape extending along the central axis Ax and is disposed in the posture coaxial with the central axis Ax. More specifically, the slider receiver 124 is inserted through the coil spring 13, and is disposed so as to be movable along the central axis Ax with respect to the first support member 121 in a state where the first support member 121 is inserted through the slider receiver 124. Here, an end of the slider receiver 124 on the distal end side Ar1 is fixed to the opening/closing mechanism 11 held in the first support member 121 by a columnar first pin Pi1 (FIG. 2) in a state where the movement along the central axis Ax with respect to the first support member 121 is permitted and the rotation about the central axis Ax is restricted.

As illustrated in FIGS. 2 to 4, the slider receiver 124 is provided with a projecting portion 1241 that protrudes from an outer peripheral surface and extends over the entire circumference in a circumferential direction surrounding the central axis Ax.

As illustrated in FIGS. 2 to 4, the slider 125 has a substantially cylindrical shape extending along the central axis Ax and is disposed in the posture coaxial with the central axis Ax. More specifically, the slider 125 is disposed so as to be movable along the central axis Ax with respect to the slider receiver 124 in a state where the slider receiver 124 is inserted through the slider 125 As described above, the slider 125 is engaged with the movable handle 3 by the pair of engaging portions 311.

Here, the coil spring 13 has a function of applying a driving force to the second grasper 9 among the first and second graspers 8 and 9 constituting the end effector 7 according to the closing operation and the opening operation of the movable handle 3 by the operator. The driving force is a driving force for opening and closing the second grasper 9 with respect to the first grasper 8. As illustrated in FIGS. 2 to 4, the coil spring 13 is disposed in a state where the slider receiver 124 is inserted through the coil spring 13 and sandwiched between the projecting portion 1241 and the slider 125.

The opening/closing mechanism 11 is a mechanism that opens and closes the second grasper 9 with respect to the first grasper 8. As illustrated in FIG. 2, 3, or 5, the opening/closing mechanism 11 includes an opening/closing connection portion 111 (FIGS. 2 and 3), an opening/closing rod 112 (FIGS. 2 and 3), and a link mechanism 113 (FIG. 5).

As illustrated in FIG. 2, the opening/closing connection portion 111 is fixed to the slider receiver 124 by the first pin Pi1, and is held in the first support member 121 so as to be movable along the central axis Ax.

The opening/closing rod 112 is an elongated member extending along the central axis Ax, and is inserted through the sheath 6. As illustrated in FIG. 2 or 3, an end of the opening/closing rod 112 on the proximal end side Ar2 protrudes to the outside of the sheath 6, is inserted through the first support member 121, and is fixed to the opening/closing connection portion 111. That is, the opening/closing rod 112 is movable along the central axis Ax together with the opening/closing connection portion 111.

The link mechanism 113 is a mechanism that connects the opening/closing rod 112 and the second grasper 9 (sixth pin Pi6).

A detailed configuration of the link mechanism 113 will be described in “Connection structure between end effector, sheath, bending mechanism, and opening/closing mechanism” described later.

The slider 125, the slider receiver 124, and the opening/closing mechanism 11 operate as described below according to the operation of the movable handle 3 by the operator.

The slider 125 is pushed toward the distal end side Ar1 along the central axis Ax by the pair of engaging portions 311 according to the closing operation of the movable handle 3 by the operator. The slider receiver 124 receives pressing force (driving force for opening and closing the second grasper 9 with respect to the first grasper 8) from the slider 125 toward the distal end side Ar1 via the coil spring 13. In addition, the opening/closing mechanism 11 moves toward the distal end side Ar1 in conjunction with the slider receiver 124. Then, the opening/closing mechanism 11 transmits the driving force to the second grasper 9. As a result, the second grasper 9 rotates about the second rotation axis Rx2 in a direction (closing direction) in proximity to the first grasper 8.

On the other hand, when the operator performs the opening operation to the movable handle 3, the slider 125, the slider receiver 124, and the opening/closing mechanism 11 operate in a direction opposite to the above direction. As a result, the second grasper 9 rotates about the second rotation axis Rx2 in a direction (opening direction) spacing apart from the first grasper 8.

The second support member 126 is a member that supports the bending operating unit 14. As illustrated in FIGS. 2 to 4, the second support member 126 includes a fitting portion 1261 and a support member body 1262.

As illustrated in FIGS. 2 to 4, the fitting portion 1261 is formed in a cylindrical shape having an outer diameter dimension substantially the same as an inner diameter dimension of the slider receiver 124, and is connected to the slider receiver 124 by being fitted in the slider receiver 124.

As illustrated in FIGS. 2 to 4, the support member body 1262 is formed in a substantially cylindrical shape having an outer diameter dimension larger than an outer diameter dimension of the slider receiver 124, and is integrally formed with the fitting portion 1261 in the posture coaxial with the fitting portion 1261. The support member body 1262 supports the bending operating unit 14 therein and is exposed to the outside of the housing body 21 from a proximal end opening portion 211 (FIG. 2) on the proximal end side Ar2 in the housing body 21.

As illustrated in FIGS. 1 to 4, the bending operating unit 14 includes a bending operating unit body 141 and a rotation converter 142 (FIGS. 2 to 4).

As illustrated in FIGS. 1 to 4, the bending operating unit body 141 has a substantially columnar shape as a whole. A columnar second pin Pi2 (FIGS. 2 to 4) is inserted on the central axis of the bending operating unit body 141. In addition, the bending operating unit body 141 is supported by the second pin Pi2 in the support member body 1262 so as to be rotatable about the second pin Pi2. In this state, the bending operating unit body 141 is located on the central axis Ax. Then, the bending operating unit body 141 receives a bending operation (operation of bending the end effector 7 with respect to the sheath 6) by the operator. By the bending operation, the bending operating unit body 141 rotates about the second pin Pi2 with respect to the support member body 1262.

As illustrated in FIGS. 2 to 4, the rotation converter 142 is connected to the bending operating unit body 141 and the bending mechanism 122. Then, the rotation converter 142 converts rotation about the second pin Pi2 according to the bending operation of the bending operating unit body 141 by the operator into rotation about the central axis Ax. That is, the rotation converter 142 rotates about the central axis Ax according to the bending operation. Examples of the rotation converter 142 include a bevel gear.

The bending mechanism 122 is a mechanism for bending the end effector 7 with respect to the sheath 6, and as illustrated in FIGS. 2 to 5, includes a rotation shaft portion 1221 (FIGS. 2 to 4), first and second drive units 1222 and 1223 (FIGS. 2 to 4), first and second bending rods 1224 and 1225 (FIGS. 2 and 3), and first and second wires 1226 and 1227 (FIG. 5).

The rotation shaft portion 1221 is a columnar elongated member extending along the central axis Ax, and is inserted through the first support member 121 in the posture coaxial with the central axis Ax. An end of the rotation shaft portion 1221 on the proximal end side Ar2 is fixed to the rotation converter 142. That is, the rotation shaft portion 1221 rotates about the central axis Ax together with the rotation converter 142 according to the bending operation of the bending operating unit body 141 by the operator.

The first and second drive units 1222 and 1223 are screwed to the rotation shaft portion 1221 by a screwing structure in which they are opposite to each other. The first and second drive units 1222 and 1223 are supported in the first support member 121 so as to be movable in opposite directions along the central axis Ax in conjunction with the rotation in the rotation shaft portion 1221 about the central axis Ax.

The first and second bending rods 1224 and 1225 are elongated members extending along the central axis Ax, and are inserted through the sheath 6. An end of the first bending rod 1224 on the proximal end side Ar2 protrudes to the outside of the sheath 6, is inserted through the first support member 121, and is fixed to the first drive unit 1222. On the other hand, an end of the second bending rod 1225 on the proximal end side Ar2 protrudes to the outside of the sheath 6, is inserted through the first support member 121, and is fixed to the second drive unit 1223. That is, the first and second bending rods 1224 and 1225 are movable along the central axis Ax together with the first and second drive units 1222 and 1223.

The first and second wires 1226 and 1227 correspond to a pair of transmission members. The first and second wires 1226 and 1227 are wires formed of a resin material or a metal material. An end of the first wire 1226 on the proximal end side Ar2 is joined to an end of the first bending rod 1224 on the distal end side Ar1 in the sheath 6. The end of the first wire 1226 on the distal end side Ar1 protrudes out of the sheath 6 and is connected to the first grasper 8. On the other hand, an end of the second wire 1227 on the proximal end side Ar2 is joined to the end of the second bending rod 1225 on the distal end side Ar1 in the sheath 6. The end of the second wire 1227 on the distal end side Ar1 protrudes out of the sheath 6 and is connected to the first grasper 8.

A detail of a connection structure between the first and second wires 1226 and 1227 and the first grasper 8 will be described in “Connection structure between end effector, sheath, bending mechanism, and opening/closing mechanism” described later. A detail of a joint structure between the first and second wires 1226 and 1227 and the first and second bending rods 1224 and 1225 will be described in “Joint structure between first and second wires and first and second bending rods” described later.

The bending mechanism 122 operates as described below according to the bending operation of the bending operating unit body 141 by the operator.

First, it is assumed that the bending operating unit body 141 is rotated (bent) in the first direction about the second pin Pi2 by the operator. In this case, the operating force is transmitted from the bending operating unit body 141 to the first and second bending rods 1224 and 1225 via the rotation converter 142, the rotation shaft portion 1221, and the first and second drive units 1222 and 1223. Then, the second bending rod 1225 moves toward the proximal end side Ar2 along the central axis Ax, and tracts the second wire 1227 toward the proximal end side Ar2. On the other hand, the first bending rod 1224 moves toward the distal end side Ar1 along the central axis Ax, and feeds the first wire 1226 toward the distal end side Ar1 according to the traction of the second wire 1227 toward the proximal end side Ar2. As a result, the end effector 7 rotates in a first bending direction Ar3 (FIG. 5) about the sixth rotation axis Rx6 with respect to the sheath 6. That is, the end effector 7 performs the bending operation.

Next, it is assumed that the bending operating unit body 141 is rotated (bent) about the second pin Pi2 by the operator in a second direction opposite to the first direction described above. In this case, contrary to the above, the first bending rod 1224 moves toward the proximal end side Ar2 along the central axis Ax, and tracts the first wire 1226 toward the proximal end side Ar2. On the other hand, contrary to the above, the second bending rod 1225 moves toward the distal end side Ar1 along the central axis Ax, and feeds the second wire 1227 toward the distal end side Ar1. As a result, the end effector 7 rotates about the sixth rotation axis Rx6 with respect to the sheath 6 in a second bending direction Ar4 (FIG. 5) opposite to the first bending direction Ar3. That is, the end effector 7 performs the bending operation.

The rotation restricting member 123 is a member that restricts rotation of the first drive unit 1222 about the central axis Ax. As illustrated in FIG. 3 or 4, the rotation restricting member 123 is fixed at a position facing the first drive unit 1222 in a direction orthogonal to the central axis Ax in the first support member 121. The first drive unit 1222 is restricted from rotating about the central axis Ax while being allowed to move along the central axis Ax with respect to the rotation restricting member 123. The rotation of the second drive unit 1223 about the central axis Ax is restricted by being abutted against the first drive unit 1222.

Connection structure between end effector, sheath, bending mechanism, and opening/closing mechanism

Next, a connection structure between the end effector 7, the sheath 6, the bending mechanism 122, and the opening/closing mechanism 11 will be described.

FIGS. 7 to 11 are diagrams illustrating the connection structure between the end effector 7, the sheath 6, the bending mechanism 122, and the opening/closing mechanism 11. Specifically, FIGS. 7 to 10 are exploded perspective views illustrating the connection structure. FIG. 11 is a cross-sectional view of the connection structure taken along a plane including the sixth rotation axis Rx6 and orthogonal to the central axis Ax.

In the first embodiment, as illustrated in FIGS. 7 to 11, a shaft body 83, a link mechanism 113, a sheath-side connection member 15 (FIGS. 7, 8, and 11), an end-effector-side connection member 16 (FIGS. 7, 8, and 11), a holding portion 17 (FIGS. 7 and 9 to 11), and a pair of retaining members 18 (FIGS. 7, 9, and 10) are used as the connection structure between the end effector 7, the sheath 6, the bending mechanism 122, and the opening/closing mechanism 11.

As illustrated in FIGS. 7 to 11, the shaft body 83 is provided at an end of the first grasper 8 on the proximal end side Ar2.

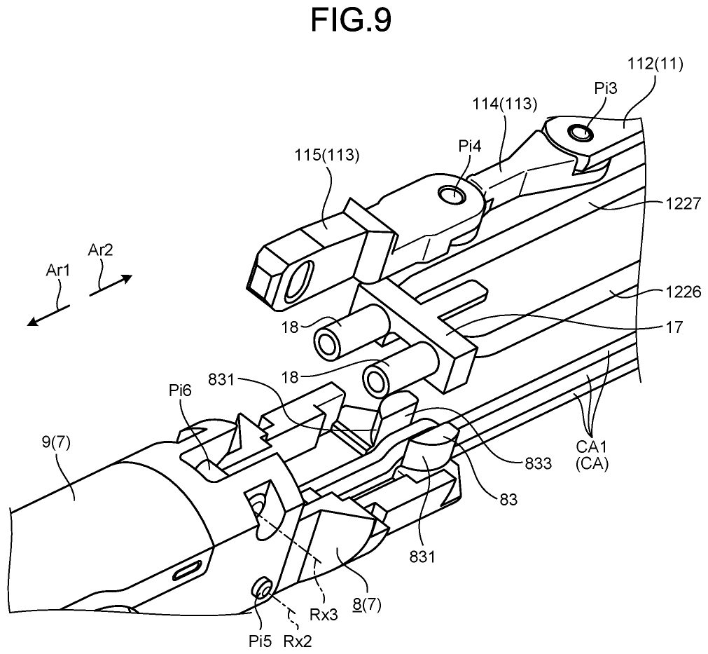

In the shaft body 83, when viewed from a direction along the sixth rotation axis Rx6, a pair of outer peripheral surfaces 831 located on a circumference of a specific circle centered on the sixth rotation axis Rx6 is provided on the upper side in FIGS. 7 and 9.

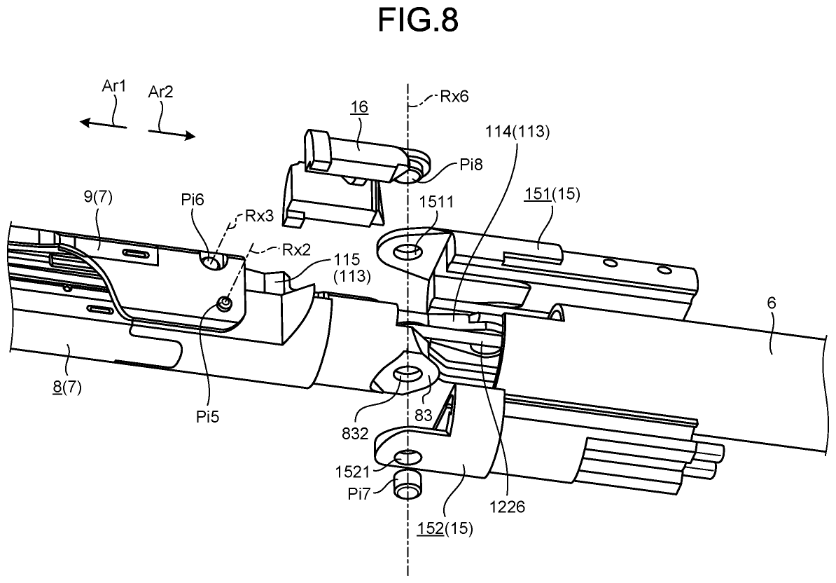

Further, in the shaft body 83, a bearing hole 832 (FIG. 8) which extends upward in FIGS. 7 and 9 and through which a columnar seventh pin Pi7 is inserted is provided on the lower side in FIGS. 7 and 9.

Furthermore, in the shaft body 83, as illustrated in FIG. 9, a passage 833 extending linearly along the longitudinal direction of the first grasper 8 is provided between the pair of outer peripheral surfaces 831. In the first embodiment, the passage 833 passes through the sixth rotation axis Rx6. The passage 833 is formed of a groove. As illustrated in FIG. 9 or 11, the four electrically conductive wires CA1 are arranged in the passage 833.

As illustrated in FIGS. 7 to 11, the link mechanism 113 includes a proximal end arm 114 and a distal end arm 115.

The proximal end arm 114 is an elongated member. The proximal end arm 114 has an end on the proximal end side Ar2 rotatably connected to the opening/closing rod 112 about a fourth rotation axis Rx4 by a columnar third pin Pi3. Here, the fourth rotation axis Rx4 is parallel to the sixth rotation axis Rx6.

The distal end arm 115 is an elongated member. In the distal end arm 115, an end on the proximal end side Ar2 is rotatably connected to an end of the proximal end arm 114 on the distal end side Ar1 about a fifth rotation axis Rx5 by a columnar fourth pin Pi4. Here, the fifth rotation axis Rx5 is parallel to the fourth rotation axis Rx4. The distal end arm 115 is connected to the second grasper 9 by the sixth pin Pi6 so as to be rotatable about the third rotation axis Rx3.

That is, in the link mechanism 113, the proximal end arm 114 and the distal end arm 115 are connected to each other so as to be movable in a specific plane orthogonal to the sixth rotation axis Rx6. Then, the link mechanism 113 transmits the driving force via the opening/closing rod 112 to the second grasper 9 while moving in the specific plane according to the bending operation of the end effector 7 with respect to the sheath 6.

As illustrated in FIG. 11, the link mechanism 113 described above is disposed in a state of being stacked along the sixth rotation axis Rx6 with respect to the shaft body 83. In other words, the link mechanism 113 and the shaft body 83 are stacked along the opening/closing direction of the second grasper 9 with respect to the first grasper 8. When viewed from the direction along the sixth rotation axis Rx6, the shaft body 83 is located between the proximal end arm 114 and the distal end arm 115.

As illustrated in FIG. 7, 8, or 11, the sheath-side connection member 15 includes a first sheath-side connection member 151 and a second sheath-side connection member 152.

The first and second sheath-side connection members 151 and 152 are fixed to an end of the sheath 6 on the distal end side Ar1 in a state where the end is sandwiched from the vertical direction in FIG. 7.

In the first sheath-side connection member 151, a bearing hole 1511 through which an eighth pin Pi8 to be described later is inserted is provided at the end on the distal end side Ar1. Similarly, in the second sheath-side connection member 152, a bearing hole 1521 through which the seventh pin Pi7 is inserted is provided at the end on the distal end side Ar1.

As illustrated in FIG. 7, 8, or 11, the end-effector-side connection member 16 is fixed to the end of the first grasper 8 on the proximal end side Ar2 in a state of covering the link mechanism 113 from the upper side in FIG. 7.

As illustrated in FIG. 8, the end-effector-side connection member 16 is provided with the columnar eighth pin Pi8 inserted through the bearing hole 1511.

The end effector 7 can perform the bending operation about the sixth rotation axis Rx6 with respect to the sheath 6 by the seventh and eighth pins Pi7 and Pi8.

Here, as illustrated in FIGS. 7 to 11, the first and second wires 1226 and 1227 are arranged along the pair of outer peripheral surfaces 831 with the shaft body 83 sandwiched between these wires. Furthermore, the first and second wires 1226 and 1227 extend toward a distal end of the end effector 7 in a state of being spaced apart from each other by a distance smaller than the diameter of the above-described specific circle constituted by the pair of outer peripheral surfaces 831. In this state, the four electrically conductive wires CA1 are arranged between the first and second wires 1226 and 1227 spaced apart from each other.

In the first embodiment, the distance smaller than the diameter of the above-described specific circle is set to a distance that maintains the state in which the first and second wires 1226 and 1227 are abutted against the pair of outer peripheral surfaces 831 even when the end effector 7 is bent, for example, to a bending angle of approximately 90° with respect to the central axis Ax in the first bending direction Ar3 or the second bending direction Ar4.

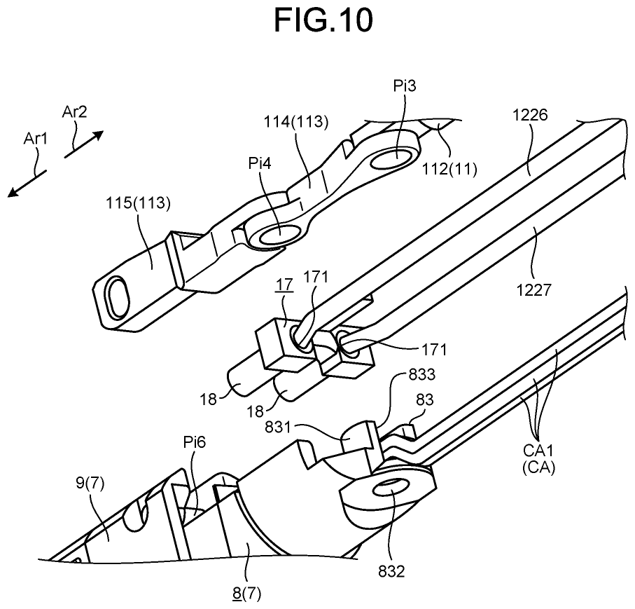

The holding portion 17 is used to fix the first and second wires 1226 and 1227 to the first grasper 8. As illustrated in FIG. 10 or 11, the holding portion 17 is provided with a pair of insertion holes 171 through which the first and second wires 1226 and 1227 are inserted. The holding portion 17 is fixed to a portion of the distal end side Ar1 relative to the shaft body 83 in the first grasper 8.

The pair of retaining members 18 is formed of a pipe and is attached to the distal ends of the first and second wires 1226 and 1227, respectively. The pair of retaining members 18 prevents the first and second wires 1226 and 1227 from coming off from the pair of insertion holes 171 in the holding portion 17.

Joint structure between first and second wires and first and second bending rods

Next, a joint structure between the first and second wires 1226 and 1227 and the first and second bending rods 1224 and 1225 will be described.

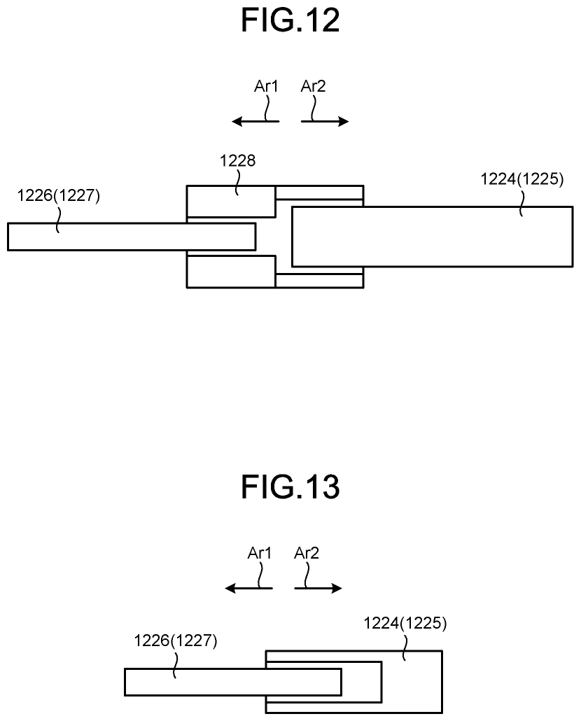

FIG. 12 is a diagram illustrating the joint structure between the first and second wires 1226 and 1227 and the first and second bending rods 1224 and 1225.

As illustrated in FIG. 12, the first wire 1226 (second wire 1227) is joined to the first bending rod 1224 (second bending rod 1225) by caulking or welding using an intermediary pipe 1228

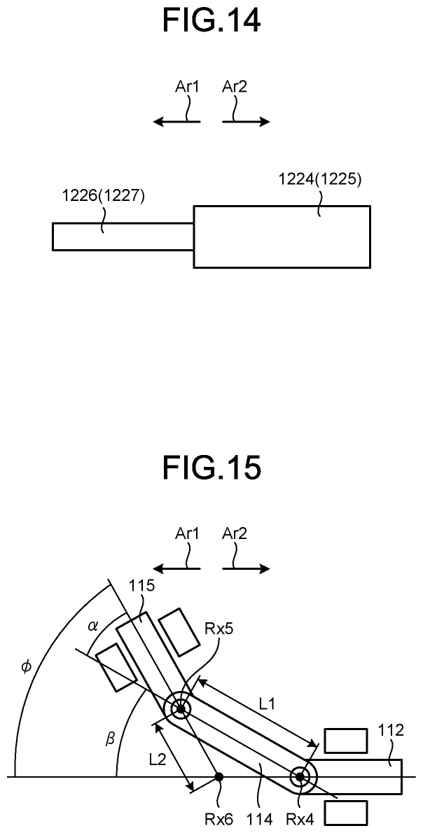

FIGS. 13 and 14 are diagrams illustrating a modification of the joint structure between the first and second wires 1226 and 1227 and the first and second bending rods 1224 and 1225.

The joint structure between the first and second wires 1226 and 1227 and the first and second bending rods 1224 and 1225 is not limited to the joint structure using the intermediary pipe 1228 described above.

For example, as illustrated in the modification of FIGS. 13 and 14, the first wire 1226 (second wire 1227) may be directly joined to the first bending rod 1224 (second bending rod 1225) by caulking or welding.

Change in Driving Force in Opening/Closing Rod and Link Mechanism

Next, a change in the driving force in the opening/closing rod 112 and the link mechanism 113 will be described.

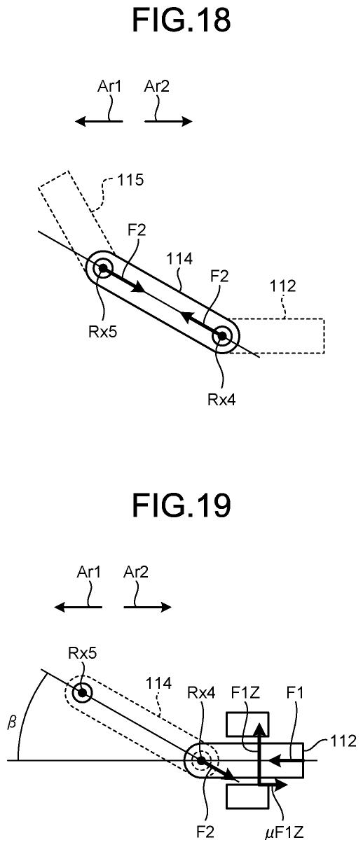

FIGS. 15 to 19 are diagrams for describing the change in driving force in the opening/closing rod 112 and the link mechanism 113.

Here, a bending angle ϕ of the end effector 7 with respect to the sheath 6 is expressed by the following formula (1).

ϕ=α+β (1)

In the formula (1), α is an angle formed by the distal end arm 115 with respect to the proximal end arm 114 (FIG. 15). β is an angle formed by the proximal end arm 114 with respect to the opening/closing rod 112 (FIG. 15).

The following formula (2) is derived from FIG. 15.

L1·sin β=L2·sin ϕ (2)

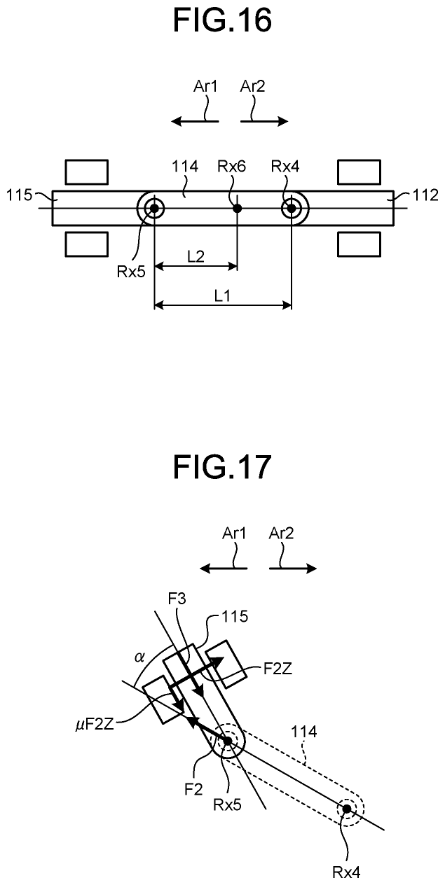

In the formula (2), L1 is a length dimension between the fourth and fifth rotation axes Rx4 and Rx5 (FIGS. 15 and 16). L2 is a length dimension between the fifth and sixth rotation axes Rx5 and Rx6 (FIGS. 15 and 16).

When the formula (2) is transformed, the following formula (3) is derived.

$$β = {sin}^{- 1} ( \frac{L 2}{L 1} \cdot sin ϕ ) \qquad ( 3 )$$

That is, from the formulas (1) and (3), it can be seen that α and β at the bending angle ϕ are determined by L2/L1.

In addition, a relationship between forces acting on the opening/closing rod 112 and the link mechanism 113 is expressed by the following formulas (4) to (7).

F3=F2·cos α−μF2Z (4)

F2Z=F2·sin α (5)

F1=F2 cos β+μF1Z (6)

F1Z=F2·sin β (7)

In the formula (4), F3 is a driving force transmitted from the distal end arm 115 to the second grasper 9 (FIG. 17). In the formulas (4) to (7), F2 is a driving force acting between the opening/closing rod 112 and the distal end arm 115 in the proximal end arm 114 (FIGS. 17 to 19). In the formulas (4) and (5), F2Z is a normal force acting on the distal end arm 115 (FIG. 17). In the formulas (4) and (6), μ is a friction coefficient. In the formula (6), F1 is a driving force transmitted to the opening/closing rod 112 (FIG. 19). In the formulas (6) and (7), F1Z is a normal force acting on the opening/closing rod 112.

When the formulas (4) to (7) are put together, the following formula (8) is derived.

$$\frac{F 3}{F 1} = \frac{( cos α - μsinα )}{( cos β + μsinβ )} \qquad ( 8 )$$

That is, it can be seen from the formula (8) that a change ratio (F3/F1) of the driving force is determined by α and β. Since α and β at the bending angle ϕ are determined by L2/L1 as described above, it can be seen that the change ratio (F3/F1) of the driving force at the bending angle ϕ is determined by L2/L1.

Here, it is assumed that the end effector 7 is configured to be bendable up to the bending angle ϕ=45°.

In this case, in order to set the change ratio (F3/F1) of the driving force to 0.75 or more and 1.25 or less, L2/L1 needs to be set to 0.2 or more and 0.9 or less.

In this case, in order to set the change ratio (F3/F1) of the driving force to 0.9 or more and 1.1 or less, L2/L1 needs to be set to 0.5 or more and 0.7 or less.

It is assumed that the end effector 7 is configured to be bendable up to the bending angle ϕ=60°.

In this case, in order to set the change ratio (F3/F1) of the driving force to 0.75 or more and 1.25 or less, L2/L1 needs to be set to 0.4 or more and 0.8 or less.

According to the first embodiment described above, the following effects are obtained.

In the treatment tool 1 according to the first embodiment, the first and second wires 1226 and 1227 are arranged along the pair of outer peripheral surfaces 831 with the shaft body 83 sandwiched between these wires. Furthermore, the first and second wires 1226 and 1227 extend toward the distal end of the end effector 7 in the state of being spaced apart from each other by the distance smaller than the diameter of the specific circle constituted by the pair of outer peripheral surfaces 831.

Thus, the length dimension when the first and second wires 1226 and 1227 are joined to the end effector 7 can be sufficiently secured. That is, joint strength of the first and second wires 1226 and 1227 to the end effector 7 can be improved. Therefore, in a state where a large force is applied to the first and second wires 1226 and 1227, it is possible to satisfactorily maintain the joining of the first and second wires 1226 and 1227 to the end effector 7.

In the treatment tool 1 according to the first embodiment, the distance smaller than the diameter of the above-described specific circle is set to the distance that maintains the state in which the first and second wires 1226 and 1227 are abutted against the pair of outer peripheral surfaces 831 even when the end effector 7 is bent, for example, to a bending angle of approximately 90° with respect to the central axis Ax in the first bending direction Ar3 or the second bending direction Ar4.

That is, even when the end effector 7 is bent to a bending angle of approximately 90°, since the first and second wires 1226 and 1227 are abutted against the pair of outer peripheral surfaces 831, it is possible to realize a structure in which sagging does not occur in the first and second wires 1226 and 1227. Thus, it is possible to avoid the end effector 7 from being easily bent by the force applied to the end effector 7 when the end effector 7 comes into contact with a living tissue or the like, and to perform treatment satisfactorily.

In the treatment tool 1 according to the first embodiment, the first and second wires 1226 and 1227 are fixed to the end effector 7 using the holding portion 17 and the retaining member 18. Thus, the first and second wires 1226 and 1227 can be joined to the end effector 7 with a simple structure.

In the treatment tool 1 according to the first embodiment, the link mechanism 113 and the shaft body 83 are stacked along the sixth rotation axis Rx6 in a state where a specific surface in which the link mechanism 113 can move is orthogonal to the sixth rotation axis Rx6. That is, the link mechanism 113 and the shaft body 83 are provided at substantially the same position in a direction along the central axis Ax.

Thus, a length of a portion on the distal end side Ar1 relative to the sixth rotation axis Rx6 can be shortened, and the distal end portion of the treatment tool 1 can be downsized. It is possible to perform fine treatment by downsizing the distal end portion of the treatment tool 1.

In the treatment tool 1 according to the first embodiment, the shaft body 83 is provided with the passage 833 through which the four electrically conductive wires CA1 are inserted. That is, the four electrically conductive wires CA1 are arranged along a shortest path passing through the sixth rotation axis Rx6 at a portion where the end effector 7 is bent.

Thus, in assembling the treatment tool 1, it is not necessary to perform a complicated operation of adjusting the length of the electrically conductive wire CA1, and assembling workability of the treatment tool can be improved.

### Second Embodiment

Next, the second embodiment will be described.

In the following description, the same reference numerals are given to the same configurations as those of the first embodiment described above, and a detailed description thereof will be omitted or simplified.

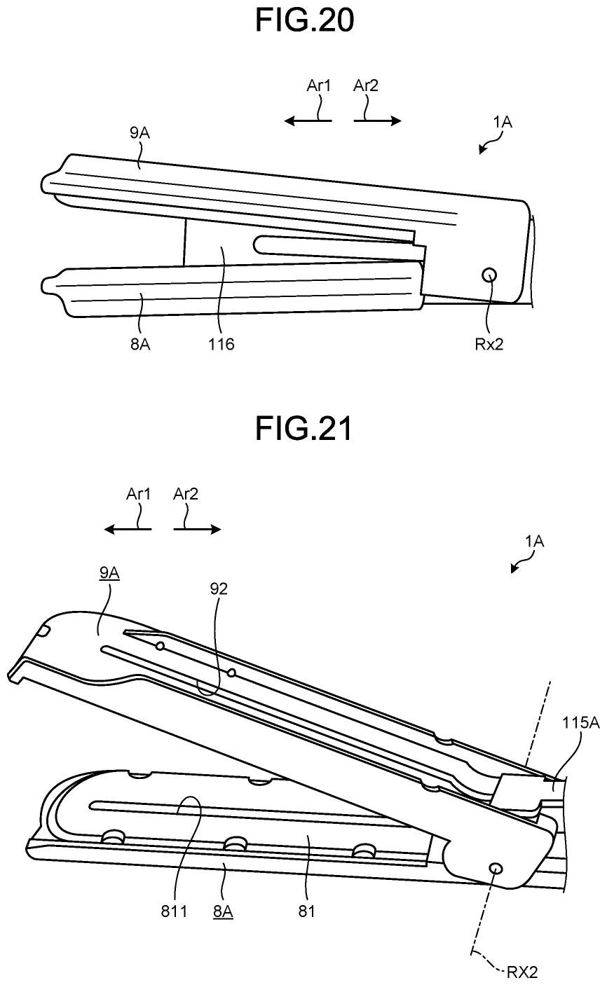

FIGS. 20 to 22 are diagrams illustrating a distal end portion of a treatment tool 1A according to the second embodiment.

In the treatment tool 1A according to the second embodiment, a cutter 116 (FIG. 20) is added to the treatment tool 1 described in the first embodiment described above.

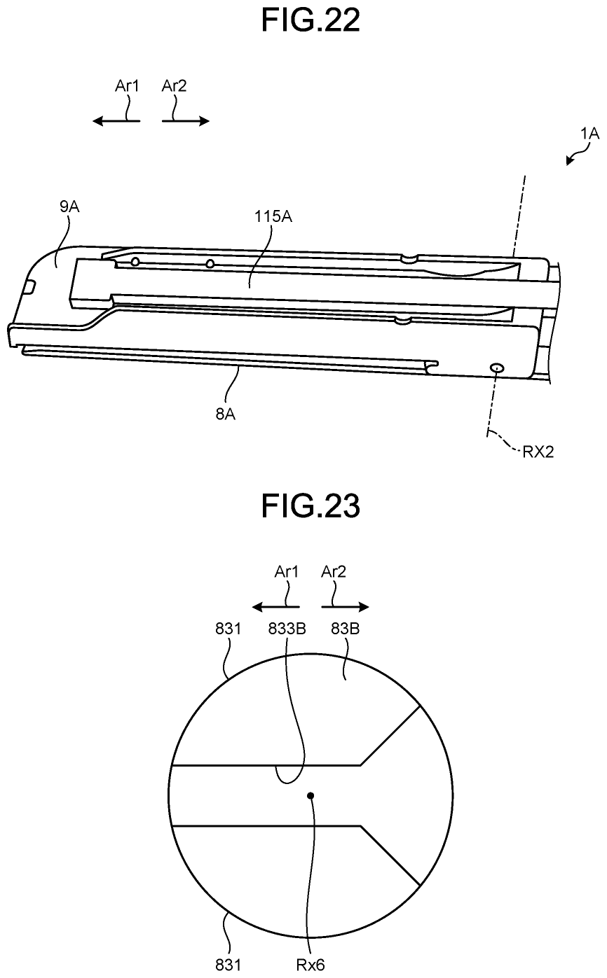

Hereinafter, for convenience of description, a distal end arm 115 according to the second embodiment will be referred to as a distal end arm 115A (FIGS. 21 and 22). First and second graspers 8 and 9 according to the second embodiment will be referred to as first and second graspers 8A and 9A, respectively.

The first grasper 8A is different from the first grasper 8 described in the first embodiment described above in the following point.

That is, as illustrated in FIG. 21, a first electrode 81 is provided with a groove 811 extending linearly along a longitudinal direction of the first grasper 8A.

The second grasper 9A is different from the second grasper 9 described in the first embodiment described above in the following point.

That is, the second grasper 9A is provided with a through hole 92 penetrating upper and lower surfaces of the second grasper 9A in FIG. 21 and linearly extending along a longitudinal direction of the second grasper 9A.

The distal end arm 115A is different from the distal end arm 115 described in the first embodiment described above in the following points.

That is, as illustrated in FIG. 21 or 22, the distal end arm 115A slides on the upper surface of the second grasper 9A toward a distal end side Ar1 or a proximal end side Ar2 in FIG. 21 according to forward and backward movement of an opening/closing rod 112 along a central axis Ax. Then, as the distal end arm 115A moves toward the distal end side Ar1, the second grasper 9A rotates about a second rotation axis Rx2 in a direction (closing direction) in proximity to the first grasper 8A. On the other hand, as the distal end arm 115A moves toward the proximal end side Ar2, the second grasper 9A rotates about the second rotation axis Rx2 in a direction (opening direction) spacing apart from the first grasper 8A.

The cutter 116 is provided at an end of the distal end arm 115A on the distal end side Ar1, and extends into the groove 811 through the through hole 92. The cutter 116 moves toward the distal end side Ar1 according to the movement of the distal end arm 115A to the distal end side Ar1. As a result, the cutter 116 incises a target site grasped between the first and second graspers 8A and 9A.

According to the second embodiment described above, effects similar to those of the first embodiment described above are obtained.

### Third Embodiment

Next, the third embodiment will be described.

In the following description, the same reference numerals are given to the same configurations as those of the first embodiment described above, and a detailed description thereof will be omitted or simplified.

FIG. 23 is a diagram illustrating a shaft body 83B according to the third embodiment.

In the shaft body 83B according to the third embodiment, the shape of a passage 833 is different from that of the shaft body 83 described in the first embodiment described above.

Hereinafter, for convenience of description, the passage 833 according to the third embodiment will be referred to as a passage 833B (FIG. 23).

In the passage 833B, a portion on a proximal end side Ar2 has a shape whose width increases toward the proximal end side Ar2 as illustrated in FIG. 23.

According to the third embodiment described above, the following effects are obtained in addition to the same effects as those of the first embodiment described above.

In the passage 833B according to the third embodiment, the portion on the proximal end side Ar2 has a shape whose width increases toward the proximal end side Ar2.

Thus, when an end effector 7 is bent, pressure applied to an electrically conductive wire CA1 from a corner portion of a side wall on the proximal end side Ar2 in the passage 833B can be reduced, and degradation of the electrically conductive wire CA1 according to the bending can be suppressed.

### Fourth Embodiment

Next, the fourth embodiment will be described.

In the following description, the same reference numerals are given to the same configurations as those of the first and third embodiments described above, and a detailed description thereof will be omitted or simplified.

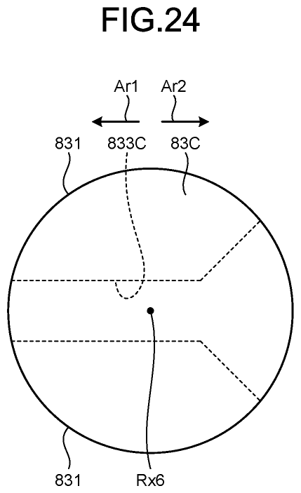

FIG. 24 is a diagram illustrating a shaft body 83C according to the fourth embodiment.

In the shaft body 83C according to the fourth embodiment, the shape of a passage 833B is different from that of the shaft body 83B described in the third embodiment described above.

Hereinafter, for convenience of description, the passage 833B according to the fourth embodiment will be referred to as a passage 833C (FIG. 24).

As illustrated in FIG. 24, the passage 833C is not formed of a groove like the passage 833B described in the third embodiment described above, but a hole.

According to the fourth embodiment described above, effects similar to those of the first and third embodiments described above are obtained.

### Fifth Embodiment

Next, the fifth embodiment will be described.

In the following description, the same reference numerals are given to the same configurations as those of the first embodiment described above, and a detailed description thereof will be omitted or simplified.

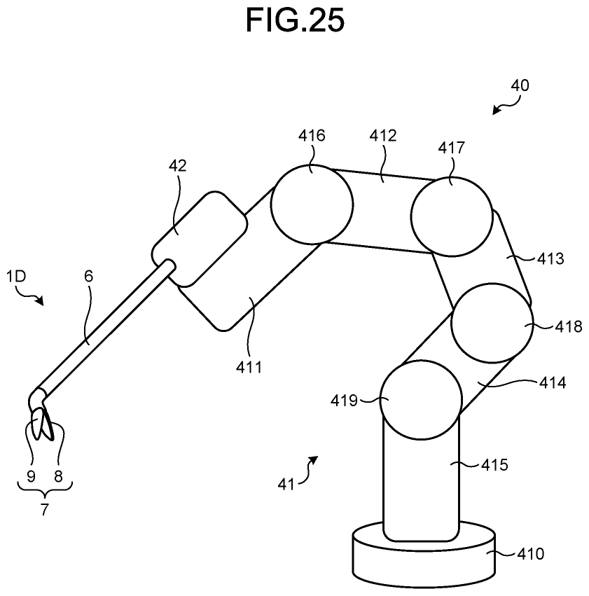

FIG. 25 is a diagram illustrating a configuration of a medical device 40 according to the fifth embodiment.

As illustrated in FIG. 25, the medical device 40 according to the fifth embodiment has a configuration in which a treatment tool 1D having a configuration different from that of the treatment tool 1 described in the first embodiment described above is supported by a robot arm 41.

As illustrated in FIG. 25, the robot arm 41 includes a base portion 410, first to fifth arm portions 411 to 415, and first to fourth joint portions 416 to 419.

The base portion 410 is installed on a floor surface or the like and supports the entire medical device 40.

The first to fifth arm portions 411 to 415 are connected in series by the first to fourth joint portions 416 to 419. Among the first to fifth arm portions 411 to 415, the fifth arm portion 415 located at a proximal end is fixed onto the base portion 410. In addition, among the first to fifth arm portions 411 to 415, the treatment tool 1D is detachably connected to the first arm portion 411 located at a distal end.

The first to fourth joint portions 416 to 419 relatively rotate a pair of mutually connected arm portions in the first to fifth arm portions 411 to 415 about mutually different axes. That is, in the fifth embodiment, the treatment tool 1D is movable in four degrees of freedom. As the robot arm 41, the degree of freedom is not limited to four, and the robot arm 41 may have another different number of degrees of freedom. That is, the number of the first to fifth arm portions 411 to 415 and the number of the first to fourth joint portions 416 to 419 are not limited to the above-described numbers, and may be other numbers.

Although not specifically illustrated, respective actuators for relatively rotating the pair of mutually connected arm portions in the first to fifth arm portions 411 to 415 are provided inside the first to fourth joint portions 416 to 419. Each actuator is driven under control by an external control device (not illustrated).

As illustrated in FIG. 25, the treatment tool 1D includes a detachable portion 42 in addition to the sheath 6, the end effector 7, the first and second bending rods 1224 and 1225, the first and second wires 1226 and 1227, the opening/closing rod 112, and the link mechanism 113 described in the first embodiment described above. In FIG. 25, the first and second bending rods 1224 and 1225, the first and second wires 1226 and 1227, the opening/closing rod 112, and the link mechanism 113 are not visible.

The detachable portion 42 is provided at a proximal end of the sheath 6 and attaches and detaches the treatment tool 1D to and from the robot arm 41 (first arm portion 411). Although not specifically illustrated, an actuator that applies driving force to the first and second bending rods 1224 and 1225 and the opening/closing rod 112 is provided inside the detachable portion 42. The actuator is driven under control by an external control device (not illustrated). As a result, the opening and closing of the second grasper 9 with respect to the first grasper 8 and the bending operation of the end effector 7 with respect to the sheath 6 are performed.

According to the fifth embodiment described above, effects similar to those of the first embodiment described above are obtained.

### Other Embodiments

Although the embodiments for carrying out the disclosure have been described so far, the disclosure should not be limited only by the first to fifth embodiments described above.

In the first to fifth embodiments described above, the high frequency energy and the thermal energy are exemplified as the treatment energy applied to a target site; however, the treatment energy is not limited thereto. As the treatment energy, at least one of high frequency energy, thermal energy, and ultrasonic energy can be adopted. Here, “applying ultrasonic energy to the target site” means applying ultrasonic vibration to the target site.

The treatment tool according to the disclosure is not limited to the configuration in which a target site is applied by applying the treatment energy to the target site, and includes forceps that only grasp the target site.

In the first to fifth embodiments described above, only the single shaft body 83, 83B, or 83C is provided, and the end effector 7 can be bent only about the sixth rotation axis Rx6; however, the disclosure is not limited thereto. The end effector 7 may be configured to be bendable about a plurality of rotation axes.

In the first to fifth embodiments described above, the link mechanism 113 is configured by two arms including the proximal end arm 114 and the distal end arm 115; however, the disclosure is not limited thereto, and the link mechanism may be configured by three or more arms.

The following configurations also belong to the technical scope of the disclosure.

(1) A treatment tool including: a tubular insertion tube at least a part of which is configured to be inserted into a body; a distal end portion which is provided at a distal end of the insertion tube and is bendable with respect to the insertion tube; a shaft body which is provided at the distal end and has an outer peripheral surface located on a circumference of a specific circle centered on a rotation axis when viewed from a direction along the rotation axis, the rotation axis allowing the distal end to be bent with respect to the insertion tube; and a pair of transmission members which are each inserted through the insertion tube, are each fixed to the distal end, and each transmit a driving force for bending the distal end with respect to the insertion tube, in which the transmission member includes a wire and a rod which is connected to a proximal end of the wire, the rod having higher rigidity than the wire.

(2) The treatment tool according to (1), in which the transmission member further includes a connection member that connects the wire and the rod.

It is assumed that the first and second bending rods 1224 and 1225 are omitted, and the first and second wires 1226 and 1227 are extended to the proximal end side Ar2 and directly fixed to the first and second drive units 1222 and 1223.

In this case, since the length dimensions of the first and second wires 1226 and 1227 increase, an amount of elongation of the first and second wires 1226 and 1227 due to a large force applied to the first and second wires 1226 and 1227 increases. As a result, sagging is likely to occur in the first and second wires 1226 and 1227, and the end effector 7 is likely to be bent by the force applied to the end effector 7 when the end effector 7 comes into contact with a living tissue or the like.

On the other hand, according to (1) and (2) described above, the length dimensions of the first and second wires 1226 and 1227 can be reduced by using the first and second bending rods 1224 and 1225. That is, it is possible to reduce the amount of elongation of the first and second wires 1226 and 1227 due to a large force applied to the first and second wires 1226 and 1227. As a result, sagging is less likely to occur in the first and second wires 1226 and 1227, and it is possible to avoid the end effector 7 from being easily bent by the force applied to the end effector 7 when the end effector 7 comes into contact with the living tissue.

(3) A treatment tool including: a tubular insertion tube at least a part of which is configured to be inserted into a body; a pair of jaws that are provided at a distal end of the insertion tube, are bendable with respect to the insertion tube, and grasp a living tissue by being opened and closed with respect to each other; a shaft body having an outer peripheral surface located on a circumference of a specific circle centered on a first pivot axis that allows the pair of jaws to be bent with respect to the insertion tube when viewed from a direction along the first pivot axis; a rod that is inserted through the insertion tube and advances and retracts along a longitudinal axis of the insertion tube; and a link mechanism that is connected to a distal end of the rod and has a plurality of arms that are connected to each other so as to be movable in a specific plane, in which the link mechanism is bendably connected to the rod about a second pivot axis and bendably connected to one of the pair of jaws about a third pivot axis, and in order to set a ratio of a second driving force transmitted from the link mechanism to one of the pair of jaws with respect to a first driving force transmitted to the rod as a ratio in a specific range, a ratio of a length L2 from the first pivot axis to the third pivot axis to a length L1 from the second pivot axis to the third pivot axis is set.

(4) The treatment tool according to (3), in which a ratio in the specific range is 0.75 or more and 1.25 or less, and a ratio of the length L2 to the length L1 is set to 0.2 or more and 0.9 or less.

(5) The treatment tool according to (3), in which a ratio in the specific range is 0.9 or more and 1.1 or less, and a ratio of the length L2 to the length L1 is set to 0.5 or more and 0.7 or less.

(6) The treatment tool according to (3), in which a ratio in the specific range is 0.75 or more and 1.25 or less, and a ratio of the length L2 to the length L1 is set to 0.4 or more and 0.8 or less.

According to (3) to (6) described above, the ratio of the second driving force to the first driving force can be easily suppressed to the ratio in the specific range, and grasping force for grasping the target site can be easily set to desired grasping force.

According to the treatment tool of the disclosure, assembling workability can be improved.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A treatment tool comprising:
a tubular insertion tube at least a part of which is configured to be inserted into a body;
an end effector that is provided at a distal end of the insertion tube and is bendable with respect to the insertion tube, the end effector being configured to apply treatment energy to a living tissue according to supplied power to treat the living tissue;
a shaft body that is provided in the end effector and has an outer peripheral surface located on a circumference of a specific circle centered on a rotation axis when viewed from a direction along the rotation axis, the rotation axis allowing the end effector to be bent with respect to the insertion tube; and
a wiring that is inserted through the insertion tube and serves as a supply path of the power,
the shaft body including a passage through which the wiring is inserted.

2. The treatment tool according to claim 1, further comprising
a pair of transmission members that are each inserted through the insertion tube and are each fixed to the end effector, each transmission member being configured to transmit a driving force for bending the end effector with respect to the insertion tube,
wherein the pair of transmission members are arranged along the outer peripheral surface in a state of sandwiching the shaft body.

3. The treatment tool according to claim 2,
wherein the pair of transmission members extend toward a distal end of the end effector in a state of being spaced apart from each other by a distance smaller than a diameter of the specific circle, and
the wiring is disposed between the pair of transmission members spaced apart from each other.

4. The treatment tool according to claim 1,
wherein the end effector includes an electrode configured to apply high frequency energy to the living tissue according to the supplied power, the high frequency energy being the treatment energy.

5. The treatment tool according to claim 1,
wherein the end effector includes a heater configured to generate heat according to the supplied power and apply thermal energy to the living tissue, the thermal energy being the treatment energy.

6. The treatment tool according to claim 1,
wherein the passage is a groove or a hole provided in the shaft body.

7. The treatment tool according to claim 1,
wherein in a portion of the passage on a side of a proximal end of the passage, a width of the passage increases toward the proximal end.
